(19) United States
(12) Patent Application Publication
(10) Pub. No.: US 2007/0249729 A1
(43) Pub. Date: Oct. 25, 2007

(54) Method of Treating Elevated Plasma Homocysteine Levels in Esrd Patients

(76) Inventors: Bradley L. Urquhart, Thornhill (CA); David J. Freeman, London (CA); Andrew A. House, London (CA); David J. Spence, London (CA)

Correspondence Address: BERESKIN AND PARR, 40 KING STREET WEST, BOX 401, TORONTO, ON, M5H 3Y2, CA

(21) Appl. No.: 10/596,479

(22) PCT Filed: Dec. 20, 2004
(86) PCT No.: PCT/CA04/02158

§ 371 Date: Mar. 29, 2007

Related U.S. Application Data
(60) Provisional application No. 60/530,237, filed on Dec. 18, 2003.

(51) Int. Cl. A61K 31/10; A61K 31/185; A61P 13/12; A61P 9/00

(52) U.S. Cl. 514/709

(57) ABSTRACT

A method of treating elevated plasma total homocysteine levels (tHc) in subjects with end stage renal disease (ESRD) is disclosed, said treatment comprising the administration of sodium 2-mercaptoethylsulfonate (MESNA) immediately prior to, or concurrently with, performing hemodialysis on said patient.

### FIELD OF THE INVENTION

The present invention relates to methods of treating elevated homocysteine levels in subjects with end stage renal disease (ESRD) using Mesna and derivatives thereof.

### BACKGROUND OF THE INVENTION

Homocysteine (Hcy) is a thiol amino acid structurally related to methionine and cysteine. Hcy is synthesized ubiquitously in mammalian cells as a product of numerous biologically important methionine dependent transmethylation reactions¹. Once synthesized intracellularly, Hcy is rapidly removed from the cell by one of three metabolic processes or it is exported to the extracellular space. Hcy can be remethylated to methionine in a recycling process by 1) the folic acid and vitamin B12 dependent enzyme methionine synthase or 2) in selected tissues by betaine homocysteine methyl transferase. Hcy may also be irreversibly catabolized to cysteine in the vitamin B6 and serine dependent transsulfuration pathway. If Hcy is unable to undergo cellular metabolism it is rapidly exported from the cell into the extracellular fluid thus maintaining low intracellular concentrations.

Numerous studies have implicated elevated total plasma Hcy (tHcy) as a graded, independent risk factor for the development of atherosclerosis^{2;3;4′}. In most patients with normal renal function, high plasma tHcy can be normalized by supplementation with the vitamins (folic acid, vitamins B₆ and B12)⁵ involved in the conversion of Hcy to methionine. Further, this intervention has been shown to inhibit the progression of plaque formations^{6′}.

Over 90% of patients with end-stage renal disease (ESRD) have elevated plasma tHcy. Although Hcy is inversely related to glomerular filtration rate (GFR)^{7;8} the actual mechanisms of its elimination remain highly debated. Of considerable clinical importance however is the fact that high dose B vitamin and folic acid supplementation lowers but consistently fails to normalize plasma tHcy in these patients^{9;10}. The leading causes of death in patients with ESRD requiring dialysis are cardiovascular related pathologies such as myocardial infarction and stroke. Despite their limited effectiveness, vitamin supplements remain the only standard treatment for elevated plasma tHcy in patients with ESRD.

A variety of tHcy lowering strategies have been tested in ESRD patients with limited success. They include 1) altering the route of vitamin administration (parenteral vs. oral)¹¹, 2) betaine supplementation¹², 3) altering the dialysis membrane pore size^{13;14}, and 4) oral N-acetylcysteine¹⁵. None of these interventions was successful in normalizing plasma tHcy in ESRD.

Plasma Hcy is 70-80% covalently bound to albumin via a disulfide bond. Although human serum albumin has 35 cysteine residues, 34 are involved in structurally important intra-chain disulfide bonds leaving only one (cys34-albumin) free and therefore capable of binding Hcy. A potential tHcy lowering strategy is to exchange Hcy on Hcy- cys34-albumin with another thiol. Oral N-acetylcysteine has been tested for this purpose and has been shown to lower plasma Hcy in patients with normal renal function however this effect was minimal when attempted in patients with ESRD^{15;16}.

Mesna (sodium 2-mercaptoethanesulfonic acid) is a thiol containing drug currently indicated for the prevention of hemorrhagic cystitis caused by oxazaphosphorine chemotherapeutic agents such as ifosfamide [U.S. Pat. No. 4,220,660]. Mesna has been on the market for several years and is considered a safe drug. The most frequent side effects reported with Mesna are nausea, vomiting, fatigue, anemia, leukopenia, asthenia, and constipation.

Most of the literature relating to Mesna describe its known use in protecting against chemotherapeutic agents [U.S. Pat. Nos. 4,220,660; 5,262,169]. U.S. Pat. No. 5,661,188 describes the use of Mesna for avoiding reperfusion injury in a donor organ following transplantation. U.S. Pat. No. 6,525,037 describes the use of Mesna for treating atherosclerosis.

Mesna has incidentally been shown to deplete plasma thiols such as Hcy and cysteine in patients undergoing chemotherapy with ifosfamide^{17;18;19}. However, those studies only relate to the possible benefits of using Mesna in cancer patients. For example, Pendyala et al.¹⁸ suggest that Mesna could potentially be used in depleting cellular glutathione levels by limiting precursors for glutathione synthesis and, therefore, improve chemotherapy efficacy.

There remains a need for effective treatment of elevated plasma thiol levels, particularly in patients refractory to the effects of B vitamins and folate, such as those with ESRD.

### SUMMARY OF THE INVENTION

Intravenous Mesna caused a rapid decrease of total plasma homocysteine (tHcy) within 30 minutes of its administration to human subjects. Mesna resulted in a decrease of post-dialysis tHcy from 18 μmol/L to 10.1 μmol/L. A large portion of the administered Mesna remained in plasma after the maximal Hcy effect occurred suggesting that a smaller dose could be used chronically when attempting to normalize plasma tHcy. Mesna was shown to be removed from plasma by dialysis and appeared in dialysate collected at various times throughout dialysis. Chronic, low dose Mesna administration represents a novel treatment option for ESRD associated hyperhomocysteinemia.

Accordingly, the present invention relates to a method of lowering elevated plasma total homocysteine (tHcy) levels in a subject with end stage renal disease comprising administering an effective amount of Mesna, or a derivative thereof, to a subject having end stage renal disease (ESRD). The invention further relates to a use of Mesna, or a derivative thereof, to lower elevated plasma total homocysteine (tHcy) levels in a subject with ESRD, as well as a use of Mesna, or a derivative thereof, to prepare a medicament to lower elevated plasma total homocysteine (tHcy) levels in a subject with ESRD.

In embodiments of the invention, by lowering the tHcy levels in the plasma of a patient with ESRD, the risk of cardiovascular-related diseases, for example myocardial infarction, stroke, thrombosis and atherosclerosis, is also lowered.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a bar graph showing the amount of free Hcy (non-protein bound) in plasma upon addition of Mesna and in a control sample in an in vitro experiment. Plasma was supplemented with reduced homocysteine (30 μmol/L) and allowed to equilibrate with protein and endogenous thiols for 72 hours. Mesna or diluent (control) was then added to the plasma and incubated at 37° C. for 15 minutes. 1 ml of plasma was pipetted into a micropartition device and centrifuged at 1500×g for 10 minutes. Free (dialyzable) Hcy was then determined in the ultrafiltrate.

FIG. 2 is a bar graph showing the effect of Mesna on the intra-dialytic change of total plasma homocysteine. Each bar represents the mean +/−SE for 3 patients. *p=0.035.

FIG. 3 is a bar graph showing the effect of Mesna on the intra-dialytic change of plasma cysteine. Each bar represents the mean +/−SE for 3 patients. *p=0.009.

FIG. 4 contains graphs showing individual patient intra-dialytic change in total plasma homocysteine with and without 5 mg/kg Mesna. A=patient 1, B=patient 2, C=patient 3.

FIG. 5 contains graphs showing individual patient intra-dialytic change in cysteine with and without 5 mg/kg Mesna. A=patient 1, B=patient 2, C=patient 3.

FIG. 6 shows pre-dialysis total plasma homocysteine concentrations (closed symbols) throughout the 14 day study period for each of the 3 patients. Post-dialysis total plasma homocysteine concentrations (open symbols) are shown for 3 dialysis sessions surrounding Mesna administration.

FIG. 7 contains graphs showing intradialytic Mesna concentrations after administration of a 5 mg/kg intravenous dose. A=patient 1, B=patient 2, C=patient 3.

FIG. 8 is a graph showing the amount of free plasma Hcy vs time for plasma that was spiked with 30 μM Hcy and allowed to bind to albumin for 72 hours. Results are mean±SE, n=6.

FIG. 9 is a graph showing the amount of free Hcy vs time in serum to which 300 μM Mesna has been added, the serum being from patients with ESRD and from healthy controls. Results are mean±SE, n=6 (control), n=5 (ESRD).

FIG. 10 is a graph showing a comparison of the efficacy of Mesna vs n-acetylcysteine (NAC) to exchange with covalently bound homocysteine. Results are mean±SE, n=6.

FIG. 11 is a graph showing the intra-dialytic plasma (n=5) and dialysate (n=2) tHcy concentrations with and without 5 mg/kg Mesna. Results are mean±SE.

FIG. 12 is a graph showing the intra-dialytic plasma (n=5) and dialysate (n=2) cysteine concentrations with and without 5 mg/kg Mesna. Results are mean±SE.

FIG. 13 is a graph showing the intra-dialytic (n=5) and dialystate (n =2) Mesna concentrations. Results are mean±SE.

FIG. 14 is a graph showing the plasma Hcy concentration after a 10 mg/kg oral dose of Mesna (at t=0) in 2 subjects with normal renal function.

FIG. 15 is a graph showing plasma Mesna concentration after a 10 mg/kg dose of Mesna (at t=0) in 2 subjects with normal renal function.

FIG. 16 is a graph showing the fractional excretion of Hcy ([CL_{Hcy}/CI_{Creat}]*100) after a 10 mg/kg oral dose of Mesna (at t=0) in 2 subjects with normal renal function.

### DETAILED DESCRIPTION OF THE INVENTION

The present inventors have shown a profound, rapid effect of Mesna on the intra-dialytic removal of plasma Hcy during hemodialysis in ESRD patients with hyperhomocysteinemia.

Accordingly, the present invention relates to a method of lowering elevated plasma total homocysteine (tHcy) levels in a subject with end stage renal disease comprising administering an effective amount of Mesna, or a derivative thereof, to a subject having end stage renal disease (ESRD). The invention further relates to a use of Mesna, or a derivative thereof, to lower elevated plasma total homocysteine (tHcy) levels in a subject with ESRD, as well as a use of Mesna, or a derivative thereof, to prepare a medicament to lower elevated plasma total homocysteine (tHcy) levels in a subject with ESRD.

The term “Mesna” refers to the drug sodium 2-mercaptoethane sulfonic acid. The derivatives of Mesna include all analogs and metabolites of Mesna including, but not limited to Mesna disulfide (or diMesna) which is the major metabolite of Mesna. It will be appreciated that Mesna or disMesna may be administered in any available form, for example as other pharmaceutically acceptable salts or solvates. In embodiments of the invention, Mesna is administered as its sodium salt. Mesna can be obtained from commercially available sources. For example, Mesna is manufactured by Baxter and distributed by Bristol Myers Squibb as Mesnex® Injection and Mesnex® Tablets. The injection contains 100 mg/ml Mesna and the tablet contains 400 mg of Mesna.

In embodiments of the invention, by lowering the tHcy levels in the plasma of a patient with ESRD, the risk of cardiovascular-related diseases, for example myocardial infarction, stroke, thrombosis and atherosclerosis, is also reduced. The term “thrombosis” includes all thrombotic events, for example, venous thrombosis, dialysis access thrombosis and thrombotic stroke.

The term total plasma homocysteine (tHcy) refers to the composite of all forms of Hcy, including, protein bound, reduced and low molecular weight mixed disulfide (with itself, cysteine or glutathione). Elevated plasma tHcy is termed hyperhomocysteinemia.

The term “elevated plasma tHcy levels” means that the amount of tHcy in the plasma of the subject is increased compared to that found in normal, healthy subjects. Typically the elevated levels of plasma tHcy is an amount which causes symptomatology associated with the disease. For example, with ESRD, elevated plasma Hcy levels would be an amount causing cardiovascular related pathologies, such as myocardial infarction, atherosclerosis, thrombosis and stroke, for example Nygard et al found that plasma levels of tHcy above 20 μmol/L were associated with a 9-fold increase in the risk of vascular disease³⁰. A meta-analysis indicated that lowering tHcy by 3 micromol/L should reduce events by 25%³¹. In general, plasma tHcy levels of greater than 12 micromoles/liter would be high.

In embodiments of the present invention, the method of lowering elevated plasma tHcy levels in a subject with ESRD comprises administering an effective amount of Mesna to a subject in need thereof and performing dialysis on the subject. The present invention also involves the use of Mesna and dialysis to lower elevated plasma tHcy levels. In embodiments of the invention, the dialysis is performed so that it results in removal of free plasma Hcy. For example, the dialysis is performed either during or subsequent to administration of Mesna. Mesna can be administered in conjunction with any form of dialysis including hemodialysis and peritoneal dialysis.

The term an “effective amount” or a “sufficient amount” of an agent as used herein is that amount sufficient to effect beneficial or desired results, including clinical results, and, as such, an “effective amount” depends upon the context in which it is being applied. For example, in the context of administering an agent that lowers elevated plasma tHcy levels in a subject with ESRD, an effective amount of an agent is, for example, an amount sufficient to achieve such a lowering of plasma tHcy levels compared to the response obtained without administration of the agent.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder.

To “decrease” or “lower” a parameter of interest, such as elevated plasma levels, is to lower the parameter of interest when compared to otherwise same conditions except for a condition or another parameter of interest, or alternatively, as compared to another conditions.

The term “subject” as used herein includes all members of the animal kingdom including humans. The subject is preferably a human.

The term “pharmaceutically acceptable” as used herein means to be compatible with the treatment of animals, in particular humans.

The term “solvate” as used herein means a compound wherein molecules of a suitable solvent are incorporated in the crystal lattice. A suitable solvent is physiologically tolerable at the dosage administered. Examples of suitable solvents are ethanol, water, oil and the like. When water is the solvent, the molecule is referred to as a “hydrate”.

Mesna is preferably formulated into pharmaceutical compositions for administration to subjects in a biologically compatible form suitable for administration in vivo. Compositions comprising Mesna for various modes of administration are known in the art. See for example U.S. Pat. No. 4,220,660 and references cited therein, the contents of which are incorporated herein by reference. In an embodiment of the invention, Mesna is administered orally or intravenously.

The dosage of the Mesna, or other pharmaceutically acceptable salts and solvates thereof, and/or compositions comprising these compounds can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the animal to be treated. One skilled in the art can determine the appropriate dosage based on the above factors. Mesna may be administered initially in a suitable dosage that may be adjusted as required, depending on the clinical response.

In an embodiment of the invention, Mesna is administered at a dosage of about 0.5-180 mg/kg per week, suitably 1.0-25 mg/kg per week, more suitably 7.5 to 15 mg/kg per week. In a further embodiment of the invention, Mesna is administered to subjects, in particular subjects with ESRD, over an extended period of time at dosages in the range of 0.5-60 mg/kg per week, suitably 1.0-25 mg/kg per week, more suitably 7.5 to 15 mg/kg per week. For example, Mesna may be administered at a dose of between about 2.5 to 5 mg/kg thrice weekly.

Mesna can be used alone or in combination with other agents that lower plasma thiol levels or in combination with other types of treatment for diseases associated with elevated plasma thiol levels. In an embodiment of the invention, Mesna is administered to ESRD patients in combination with B vitamins and/or folic acid.

The following non-limiting examples are illustrative of the present invention:

### EXAMPLES

The objective of the experiment was to determine whether a single intravenous dose of Mesna is capable of increasing the intra-dialytic clearance of Hcy in patients with ESRD. The inventors also evaluated Mesna's intra-dialytic kinetics. It was hypothesized that Mesna administration would cause thiol exchange resulting in an acute decrease in plasma tHcy and cysteine. The inventors further hypothesized that Mesna would be dialytically removed although the removal will be limited by its protein binding.

Methods

Subjects

3 vitamin supplemented (Replavite, Landmark Medical Systems Inc. Unionville, Ontario) maintenance hemodialysis patients (2 males, 1 female) were recruited from the dialysis unit at London Health Sciences Centre—University Campus. Inclusion criteria were any vitamin supplemented patients on thrice weekly hemodialysis for at least 90 days who were willing to sign a letter of informed consent. Patients were excluded from the study if they had serum albumin≦30 g/L. The study protocol was approved by the human subject research ethics board at the University of Western Ontario. Patients were dialyzed with Fresenius Optiflux hollow fiber dialyzers (Fresenius AG, Bad Homburg, Germany).

Study Protocol—In Vitro

30 ml of blood (EDTA) was obtained from a healthy subject with normal renal function. The blood was immediately centrifuged at 1500×g and the plasma was separated. D,L Homocysteine was then added to the plasma to a final concentration of 30 μmol/L. The sample was incubated at 4° C. for 72 hours to allow the homocysteine to covalently bind to protein and other thiols. Mesna was added to 1 ml of plasma to a final concentration of 305 μmol/L. An equal volume of diluent was added to 1 ml of plasma as a control. The plasma (1 ml) was then incubated at 37° C. for 15 minutes after which it was pipetted into an Amicon (Amicon Canada, Oakville, Ontario) type YMT micropartition system for separation of free from protein bound solutes. The micropartition system was centrifuged at 1500 X g for 10 minutes and the ultrafiltrate obtained was stored at −20° C. until analysis.

Study Protocol—In Vivo

To assess baseline total plasma Hcy concentrations, 5 ml blood samples (EDTA) were drawn pre- and post-dialysis for all 7 dialysis sessions during the 14 day study period. On day 5 (corresponding to the third dialysis session) blood samples were also drawn at various times throughout the dialysis treatment (0.5, 1.0, 2.0 and 3.0 hours) to determine baseline kinetic data. Undiluted ultrafiltrate was obtained from 2 patients by stopping the dialysate flow while maintaining the patient on ultrafiltration. Once 200 ml of dialysate had been displaced from the dialyzer, an undiluted ultrafiltrate sample was drawn from a port specially installed for the study. On the treatment day (a mid-week dialysis session corresponding to day 7 of the study), a single 5 mg/kg dose of Mesna was infused directly into the venous return on the dialysis machine over 5 minutes. Blood samples were drawn pre-dialysis, 4 minutes after Mesna administration, 0.5, 1.0, 2.0, 3.0 hours and post-dialysis. Undiluted ultrafiltrate was also collected from 2 of the patients as described above. Pre- and post-dialysis blood samples were also drawn on each of the 3 subsequent dialysis treatments (corresponding to days 9, 12 and 14) to determine the duration of Mesna's action and how long it remains in the blood. All blood samples were immediately placed on ice and centrifuged within 30 minutes at 1500×g for 10 minutes. Plasma and ultrafiltrate were stored at −20° C. until analysis.

Analytical Procedures

Total plasma Hcy and other low molecular weight thiols were measured by HPLC-FD after pre-column derivitization as described by Jacobsen et al.²⁰ Briefly, disulfide bonds were reduced with 1.43 M sodium borohydride. Excess reducing agent was consumed by the addition of 1.2 M HCl after which thiols were derivitized with monobromobimane. Protein was precipitated with 1.5 M perchloric acid and the sample pH was raised to ˜3.5 with a solution of 2M citrate and 10 N NaOH.

Thiol amino acids were separated on a Waters Nova-Pak C18 (150×3.9 mm, 5μ) column. The mobile phase consisted of 4% acetonitrile/25 mM ammonium formate buffer, pH=3.8. Since Mesna is also a low molecular weight thiol it can also be measured as a bimane derivative, although it is very late eluting when the above mentioned chromatographic conditions are used. Therefore, the inventors modified the chromoatographic conditions such that 4% acetonitrile was run for 7 minutes and then a gradient of 4% to 70% acetonitrile was used from 7 to 20 minutes. The retention time of Mesna under these conditions was 13 minutes.

Statistical Methods

Results are shown as mean +/−SD. Intra-dialytic changes in tHcy and cysteine concentrations were analyzed by a paired students t-test. A p value <0.05 was considered significant.

### Example 1

In Vitro Study

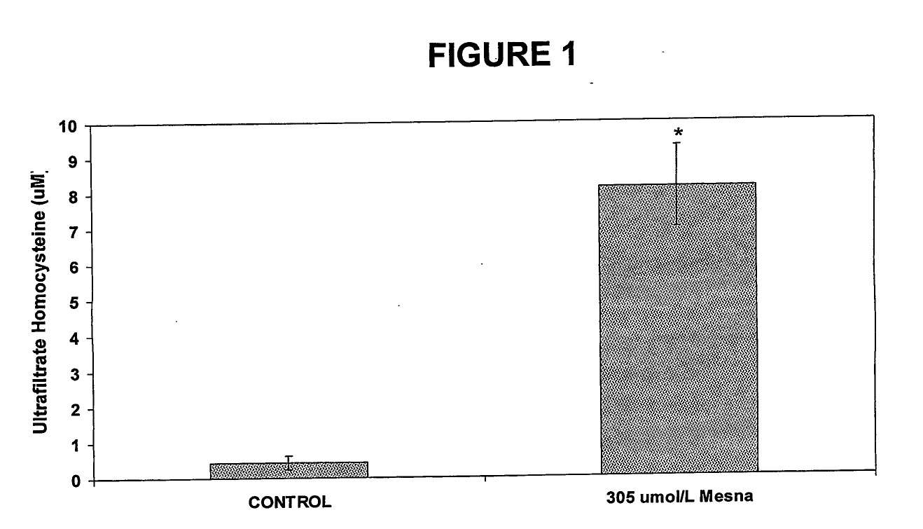

Mesna treatment caused a rapid change in the distribution of plasma Hcy from protein bound to free as evidenced by the amount measured in the ultrafiltrate. At time 0 (before addition of Mesna) only 0.5 μmol/L (1.7% of the total plasma Hcy) was in the free form. After only a 15 minute incubation with 305 μmol/L Mesna, 8.2 μmol/L (27.2% of the total) of Hcy was in the free dialyzable form. There was no change in the amount of free Hcy in the control sample at the end of the incubation (see FIG. 1).

### Example 2

In Vivo Study

All 3 patients had hyperhomocysteinemia (total plasma Hcy>15 μmol/L) and elevated plasma cysteine. The baseline pre-dialysis total plasma Hcy and cysteine on day 0 of the study were 24.4+/−6.1 μmol/L and 358.6+/−35.8 μmol/L respectively. All patient characteristics are shown in table 1.

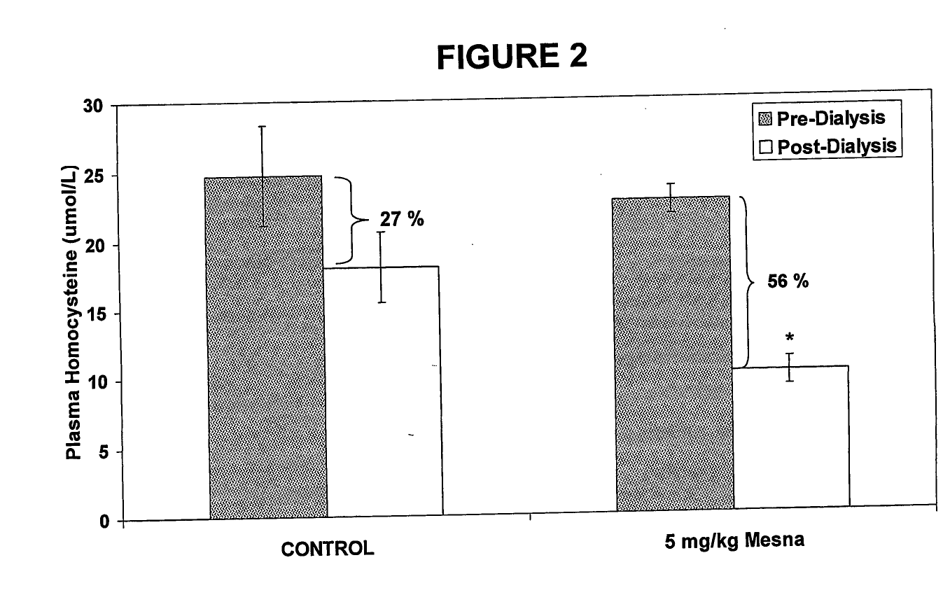

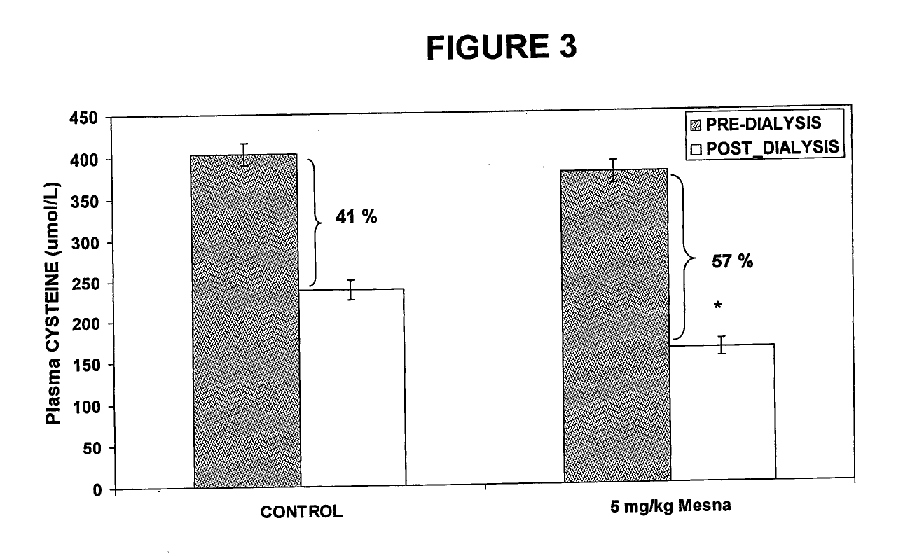

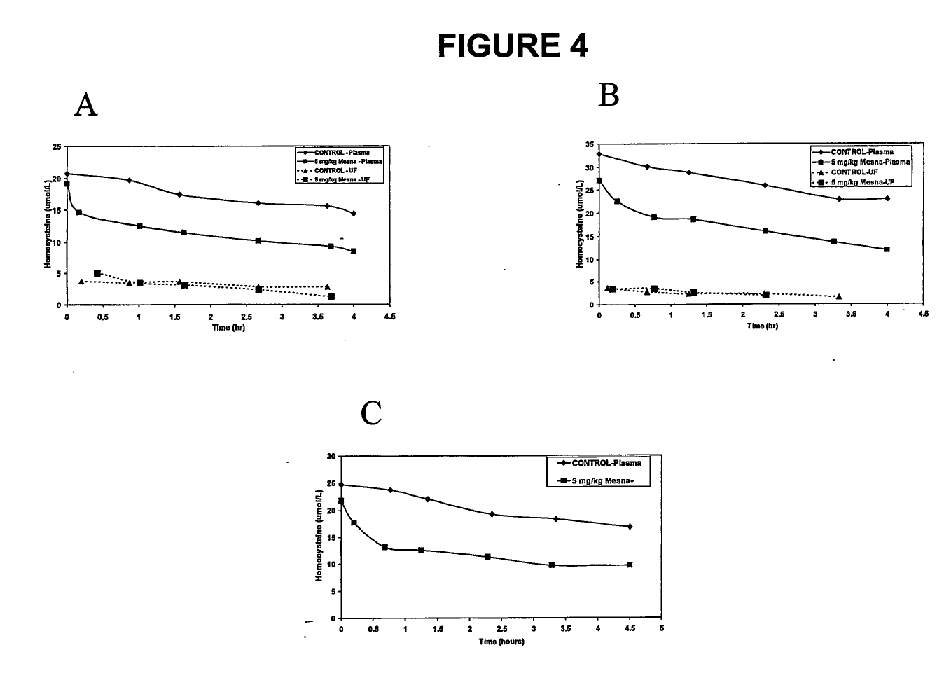

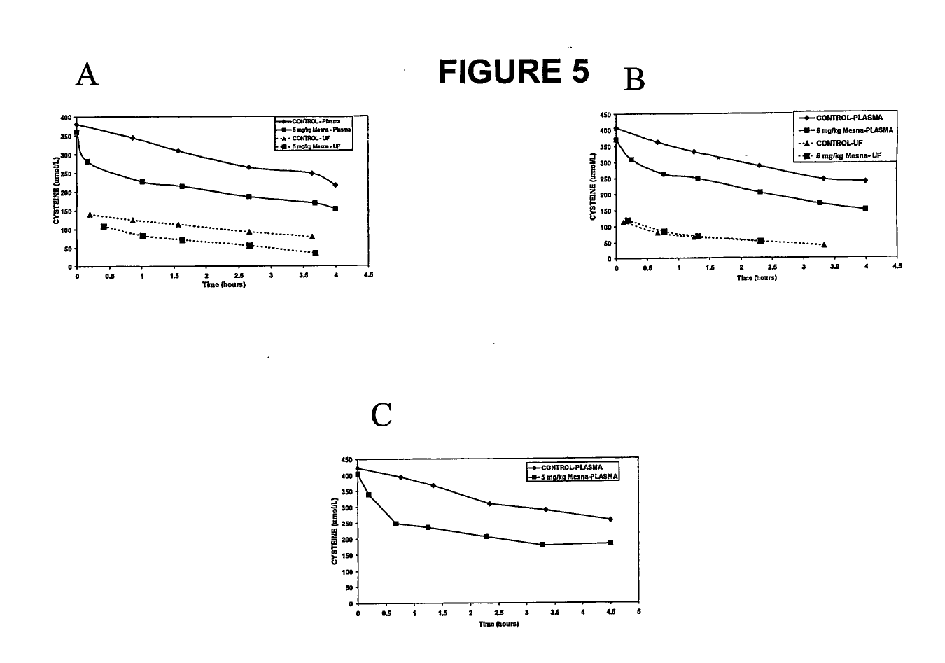

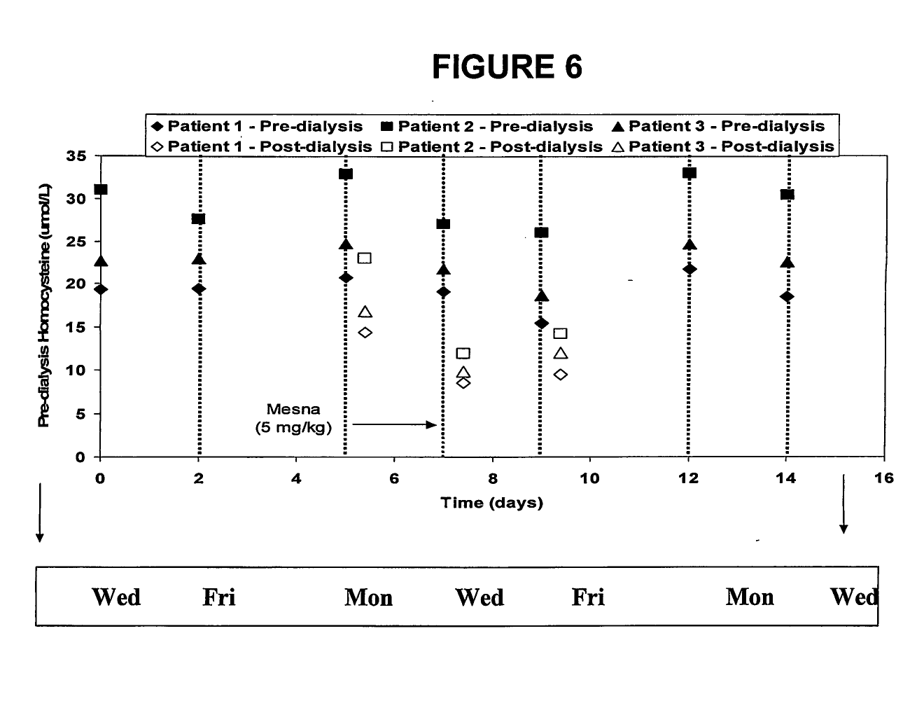

Total plasma Hcy and cysteine both decreased post-dialysis (27% and 41% respectively) in the absence of Mesna treatment. Treatment with Mesna caused a significant decrease in the post-dialysis tHcy (p=0.035) and cysteine (p=0.009) as shown in FIGS. 2 and 3. Further, the Mesna effect was rapid and short-lived occurring during the first 30 minutes of dialysis (see FIGS. 4 and 5). The pre-dialysis tHcy on subsequent cycles remained lower in 2 patients (see FIG. 6, day 9). By the second cycle (day 12) pre-dialysis plasma Hcy had returned to baseline in all patients.

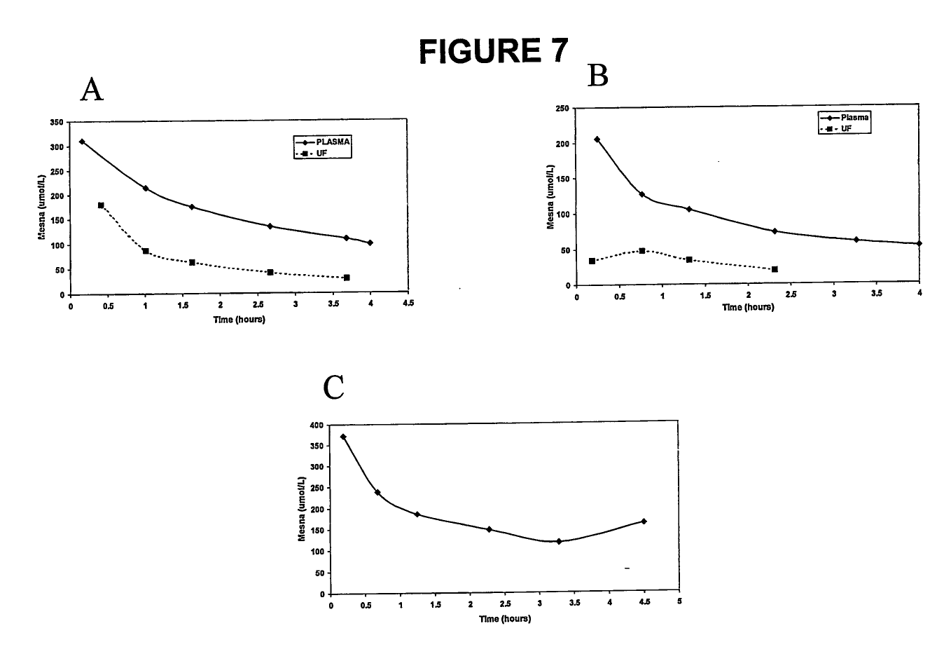

Mesna concentrations in plasma and UF (where applicable) were monitored throughout the test dialysis and on the 3 dialysis treatments thereafter to determine whether Mesna is dialytically cleared and how long it remains in the blood. The intra-dialytic removal of Mesna is shown in FIG. 7. Plasma Mesna decreased from 295.9+/−83.7 μmol/L shortly after administration to 89.7+/−32.5 μmol/L post-dialysis. Mesna appeared in the UF of both patients for which it was collected. Mesna was still present in the plasma of all three patients at the time of the next dialysis treatment (day 9). The plasma Mesna concentration at the end of the study period (day 14) was 13.4+/−12.1 μmol/L.

### Example 3

Tolerability of Mesna

Two patients indicated they had a feeling of mild nausea. One patient's symptoms were present almost immediately after dosing and the other's symptoms did not present until ˜3.5 hours after dosing. Both patient's symptoms were rapidly (within 1 minute) reversed after administration of dimenhydrinate.

### Discussion for Examples 1-3

All 3 patients with ESRD tested in this pilot study had hyperhomocysteinemia consistent with numerous literature reports^{21;22;23;24;25}. Over 90% of patients with ESRD have elevated total plasma Hcy and the leading causes of death are cardiovascular related pathologies. The mechanism of Hcy accumulation in renal disease remains unknown and highly debated. For this reason treatment options for decreasing total plasma Hcy in ESRD are limited to supplementation with water soluble vitamins (folic acid, and vitamins B6 and B12) as well as daily or nocturnal hemodialysis²⁹ and possibly variations in the dialysis membrane¹⁴. Vitamin supplementation consistently fails to normalize total plasma Hcy in patients with ESRD even in pharmacological doses¹⁰;. Due to the inability to normalize total plasma Hcy in patients with ESRD, it is unknown whether normalizing total plasma Hcy will decrease morbidity and mortality.

The present inventors have demonstrated a profound, rapid effect of Mesna on the intra-dialytic removal of plasma Hcy during hemodialysis. The effects of oral and intravenous Mesna and its metabolite diMesna on plasma thiols have been well characterized in patients undergoing chemotherapy with ifosfamidel^{17;18;19;26}. Intravenous Mesna administration for 5 days to patients undergoing cancer chemotherapy was found to cause a decrease in several plasma thiols (eg Hcy decreased from 12.3 μmol/L to 1.4 μmol/L)¹⁷. Mesna is thought to act by exchanging with thiols bound to albumin thus enhancing their renal excretion. The present studies confirm this mechanism of action by showing that Mesna exchanges with Hcy on albumin yielding a larger fraction of free dialyzable Hcy. Mesna's ability to exchange with thiols lies in the pKa of its thiol moiety. Whereas the thiols on homocysteine and cysteine have pKa's of 8.7 and 8.15 respectively, the pKa of the thiol on Mesna is 9.1. This results in a more thermodynamically stable bond with Mesna and the single available reactive thiol on albumin (cys34-albumin).

The present in vitro results suggest Mesna at current therapeuctic levels will cause 27% of albumin bound Hcy to dissociate and become freely dialyzable as Hcy-cysteine, Hcy-Hcy, Hcy-Mesna or homocysteine. The in vivo results directly support those obtained in vitro. The introduction of Mesna (a single intravenous 5 mg/kg dose) during dialysis caused total plasma Hcy to decrease an additional 29% compared to dialysis alone. Post-dialysis tHcy concentrations in the absence of treatment were 18 μmol/L whereas they deceased to 10.1 μmol/L with Mesna treatment. No increase in UF Hcy was observed in any sample following Mesna treatment. In retrospect this is presumably due to inability to capture the rapid exchange process with the inventors' sampling protocol. Further in vitro studies with plasma in the laboratory have revealed that maximal exchange occurs within 2 minutes of Mesna addition.

Pre-dialysis tHcy concentrations were followed for 3 dialysis sessions after Mesna treatment. Pre-dialysis tHcy concentrations were still lower at the time of the next dialysis session (day 9). Although this difference did not reach statistical significance, the deleterious effects of elevated plasma tHcy are graded and even small decreases in plasma tHcy represent clinically important reduction in risk of cardiovascular problems. Patients 1 and 3 had steady baseline plasma tHcy concentrations on the days before Mesna administration. The slight increase in baseline pre-dialysis tHcy between days 2 and 5 represents 3 days between dialysis sessions and hence a longer time for Hcy to accumulate. Patient 2 had variable plasma tHcy in the days leading up to Mesna administration. It is unknown whether this was due to non-compliance with vitamin supplements. In patients 1 and 3 pre-dialysis plasma tHcy was 3.7 and 3.1 μmol/L less on study day 9 than on the day Mesna was administered (day 7). Patient 2 showed only a 1.1 μmol/L decrease in pre-dialysis tHcy on day 9.

The present inventors have demonstrated that a single intravenous dose of Mesna causes a rapid decrease in plasma tHcy presumably by increasing the intra-dialytic removal of Hcy. If Mesna is administered chronically at an appropriate dosing interval in combination with routine vitamin supplements, it is submitted that pre-dialysis plasma tHcy will normalize. Interestingly, high residual Mesna concentrations are found in the blood after the maximal effect on Hcy levels has been achieved. This suggests that free Mesna is removed during dialysis and the remaining protein-bound Mesna offers no further displacement of Hcy. Whether lower doses of Mesna would be equally efficacious at exchanging with Hcy-albumin remains to be determined. Although Mesna is considered to be a safe drug, careful monitoring for side effects during chronic administration is necessary.

The effect of intravenous Mesna on plasma cysteine was similar to that of tHcy. Mesna caused a rapid decrease in plasma cysteine within the first half hour of dialysis after which the Mesna treated and control plasma cysteine paralleled each other. It has been suggested by other groups that elevated plasma cysteine inhibits Hcy transsulfuration. Elevated plasma cysteine has been shown to down-regulate cystathionine beta synthase (CBS)¹⁷, the enzyme responsible for first step of the irreversible catabolism of Hcy to cysteine. It may be possible that chronically administered Mesna causes a sustained decrease in plasma cysteine concentrations and if decreased plasma cysteine would lead to increased flux of Hcy through the transsulfuration pathway resulting in an even further decrease in plasma tHcy.

Previous attempts to lower plasma tHcy using compounds known to undergo thiol exchange reactions have had minimal success. A recent report by Friedman et al. attempted to use oral N-acetylcysteine to decrease plasma tHcy concentrations in patients on hemodialysis¹⁵. The results of the Friedman et al. study showed a small, non-significant decrease in pre-dialysis plasma tHcy after 4 weeks of treatment. The present results suggest that thiol exchange reactions occur very rapidly and that drugs targeted to decrease plasma tHcy in patients on hemodialysis should be administered at the beginning of dialysis. The kidney represents a large source of glutathione reductase, the enzyme responsible for reducing many oxidized thiols including diMesna. Patients with ESRD will not have the same renal capacity to reduce inactive diMesna to Mesna and therefore careful precaution must be taken to administer the drug in its reduced form. However, the isolated perfused rat liver has been shown to reduce diMesna to Mesna²⁸ and presumably the human liver can do the same but this remains to be evaluated. Theoretically pre-dialysis diMesna administration would also be capable of increasing the fraction of dialyzable Hcy by thiol exchange. Effective lowering of plasma tHcy by thiol exchange relies heavily on dialysis in ESRD. Therefore reduction of diMesna, (whether given directly or resulting from oxidation of Mesna) by the liver between dialysis sessions is not expected to cause a further decrease in plasma tHcy.

The manufacturer of Mesna does not recommend dosage adjustment for patients with renal failure who are receiving ifosfamide. The recommended dose of Mesna to prevent hemorrhagic cystitis is between 10-12 mg/kg. A dose of 5 mg/kg was chosen in the present study because there is very little known about the dialysis of Mesna. To the best of the knowledge of the present inventors this is the first report that Mesna is removed by dialysis. Similar to Hcy and cysteine, plasma Mesna concentrations rapidly decreased at the beginning of dialysis and appeared to obey first order elimination. Mesna was measured in the UF of 2 patients enrolled in this study. In patient 1 UF Mesna was less than plasma Mesna at all time points measured. This discrepancy is likely due to the protein binding of Mesna and it is interesting to note that the decrease in plasma Mesna is mirrored by the Mesna recovered in the UF. In patient 2 the first UF collection had relatively little Mesna and did not appear to follow the plasma Mesna. For this patient, the UF sample was collected during the last minute of Mesna infusion to try to determine if a larger concentration of Hcy was being removed early and/or during Mesna administration. Unfortunately, it appears that this sample was drawn too early as Mesna administration was not yet complete and the drug did not have time to distribute and have its effect.

Two of the patients experienced nausea which is a concern if Mesna is to be used chronically to lower plasma tHcy. One patient's nausea was present almost immediately after administration of the drug. Interestingly, the second patient had no feeling of nausea until ˜3.5 hours after Mesna administration at which time s/he had relatively low (˜60 μmol/L) plasma Mesna. Importantly, feelings of nausea were rapidly reversed by intravenous administration of dimenhydrinate. It is believed that lower doses of Mesna would be equally efficacious and reduce the incidence of nausea.

### Example 4

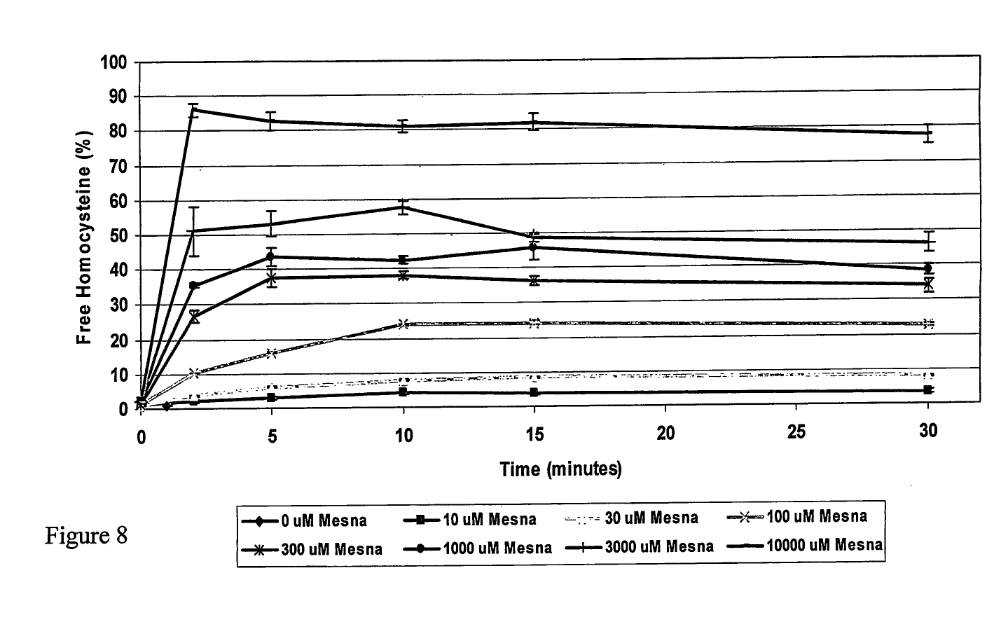

To determine the efficacy of Mesna in vitro a thiol exchange assay was developed. This assay evaluates the ability of thiol-containing drugs to exchange with protein bound Hcy and other thiols yielding a larger free, dialyzable fraction of Hcy. 60 mL of blood was drawn from 6 healthy subjects and plasma was obtained by centrifugation at 1500×g. D,L Homocysteine was then added to each plasma sample to a final concentration of 30 μM. The samples were allowed to equilibrate at 4° C. for 72 hours to allow covalent, disulfide bonding of Hcy to plasma proteins. To conduct each assay, 380 μL of the aforementioned plasma was pipetted into a 1.7 mL polypropylene snap-cap tube. 20 uL of Mesna (diluted in 4 mM ethylenediaminetetraacetic acid disodium salt (EDTA), pH=7) was then added to the sample to final concentrations ranging from 0 μM (EDTA vehicle) to 10 000 μM. The sample was incubated for 30 minutes at 37° C. 50 μL aliquots were removed at 0, 2, 5, 10, 15 and 30 at which time plasma proteins were precipitated with 25 μL of 15% sulphosalicylic acid. 13 μL of citrate/NaOH (1M:5M respectively) was added to raise the pH to approximately 7.50 μL of the resulting solution was then assayed for Hcy by a slight modification to the method of Jacobsen et al.²⁰ The results are summarized in FIG. 8. Results for each concentration of Mesna are mean +/−SE, n=6. Mesna caused a concentration dependent increase in the fraction of free dialyzable Hcy, in vitro. These in vitro results suggest that Mesna administration to patients with ESRD would result in decreased plasma tHcy by exchanging with protein bound Hcy ultimately leading to increased dialytic clearance.

### Example 5

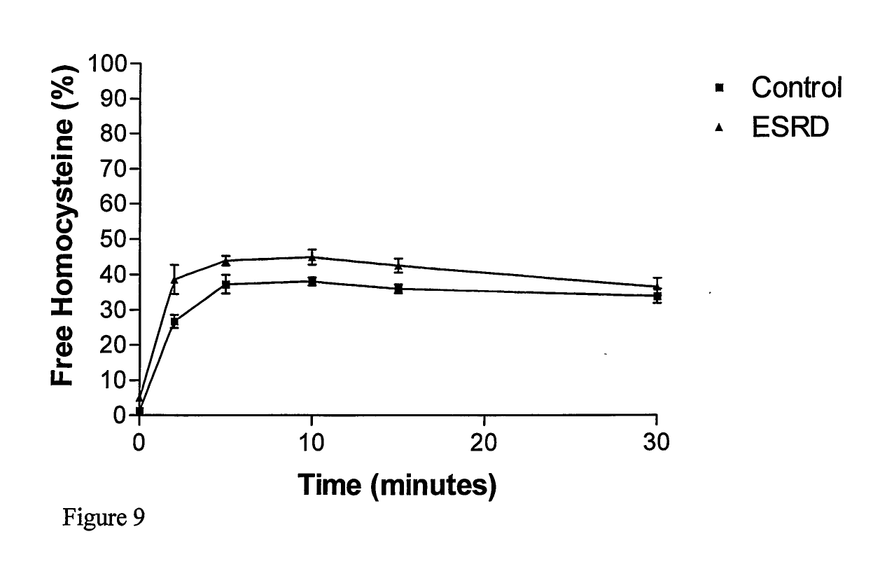

Numerous compounds that may modulate protein binding and thiol exchange accumulate in the serum of patients with end-stage renal disease (ESRD). To verify that none of these compounds alter Mesna's exchange with protein bound Hcy, a single concentration of Mesna (300 μM) was tested in a thiol exchange assay (described in Example 4) in serum from patients with ESRD or healthy controls. Results are mean +/−SE, n=6 (control), n=5 (ESRD) and are presented in FIG. 9. There was no significant difference in the AUC comparing thiol exchange from ESRD serum vs. healthy controls (P>0.05). There was more free Hcy at t=0 in the ESRD group than the healthy control group likely representing disturbed binding to protein in uremic serum however this did not translate into decreased efficacy of Mesna.

### Example 6

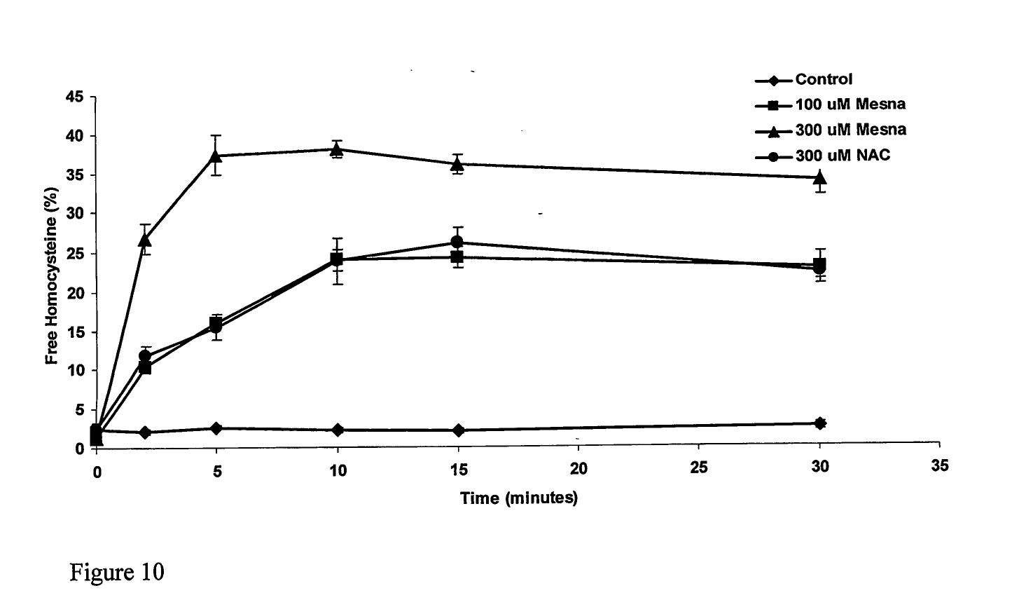

The in vitro thiol exchange potential of Mesna was compared with another thiol-containing drug, n-acetylcysteine (NAC) by the thiol exchange assay described in Example 4. 300 μM NAC was compared with 150 and 300 μM Mesna in the plasma of healthy subjects. Results are mean +/−SE, n=6 and are presented in FIG. 10. The figure indicates that 300 μM Mesna was more efficacious than 300 μM NAC (AUC for Mesna 1023+/−86.0 vs. 642+/−49.7 for NAC, P<0.001).

### Example 7

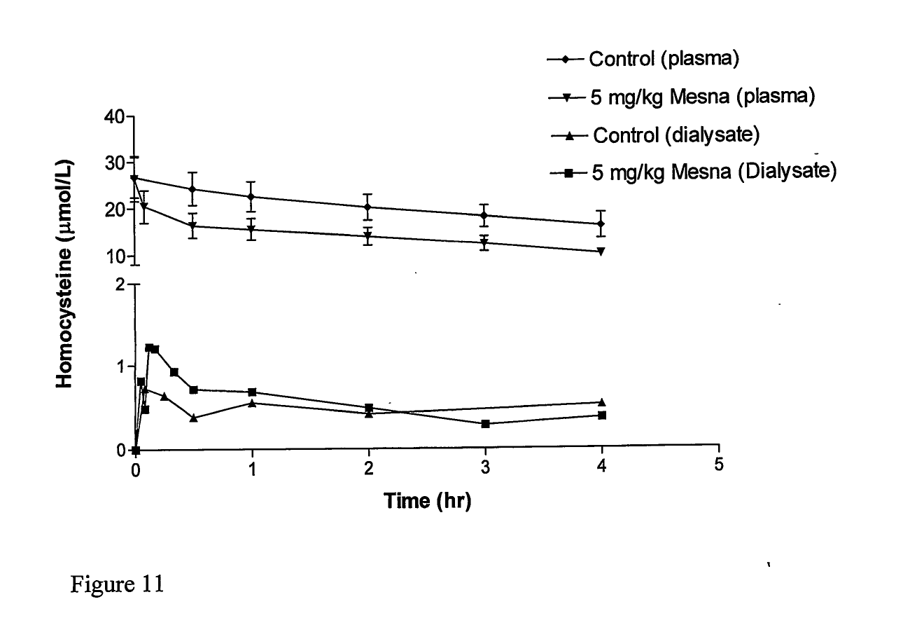

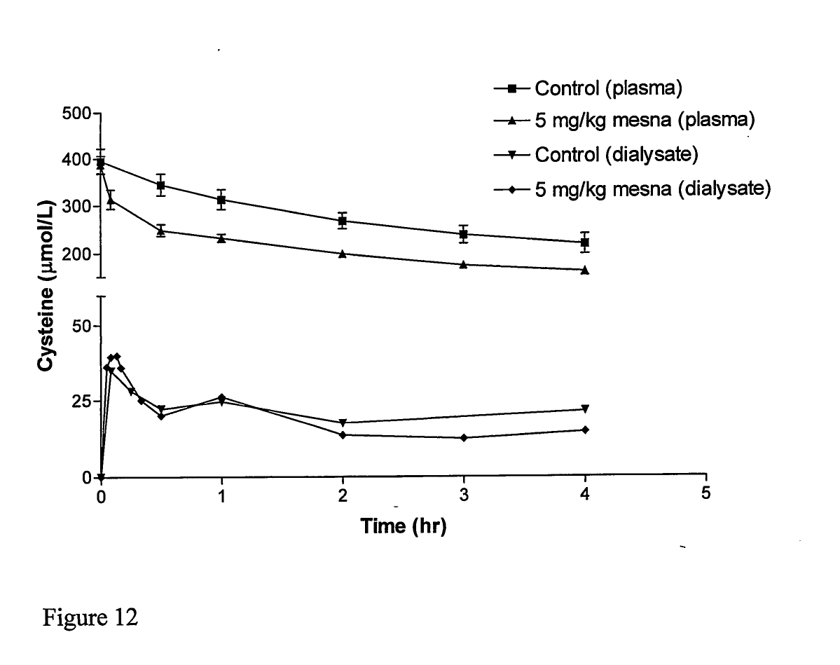

To evaluate the in vivo effect of Mesna on plasma tHcy, 5 maintenance hemodialysis patients were recruited to participate in a single dose pilot study. Blood samples were drawn at selected intervals during a mid-week dialysis session during which no Mesna was given. One week later, subjects received a single, 5 mg/kg, pre-dialysis, intravenous dose of Mesna and blood samples were drawn throughout the dialysis session. Dialysate samples were also collected from 2 patients. Total Hcy and cysteine were measured in plasma and dialysate by the modified method of Jacobsen et al.²⁰ Mesna caused a profound, rapid decrease in plasma tHcy and cysteine (see FIG. 11 and 12). There was a slight increase in dialysate Hcy with Mesna treatment compared to control. Post-dialysis, plasma tHcy and cysteine were significantly decreased with Mesna compared to control (tHcy 11.6+/−1.7 μM vs. 17.4+/−2.5 μM, P=0.03, cysteine 165.1+/−7.4 μM vs. 219.8+/−16.1 μM, P=0.02). A pre-dialysis plasma sample was also drawn before the next dialysis session 2 days later. Plasma tHcy was 2.3 μM lower with Mesna than control (P=0.047) indicating a residual effect of Mesna on tHcy concentrations.

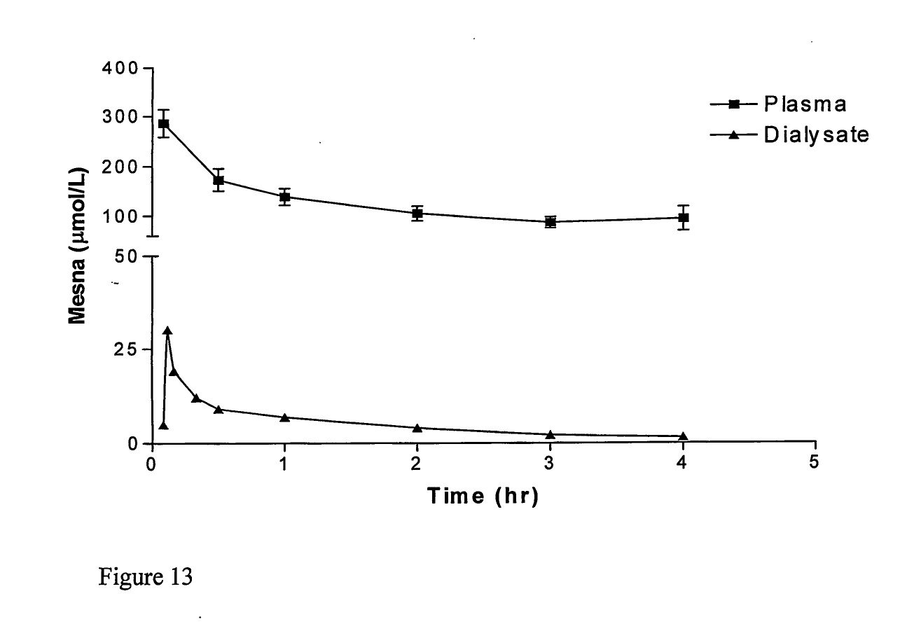

Concentrations of Mesna were also measured in plasma (n=5) and dialysate (n=2) (see FIG. 13). Mesna decreased from 286.0+/−27.6 μM at the beginning of dialysis to 91.3+/−19.3 μM post-dialysis. Further, Mesna appeared to follow first order elimination and was recovered in the dialysate indicating it is dialytically cleared.

### Example 8

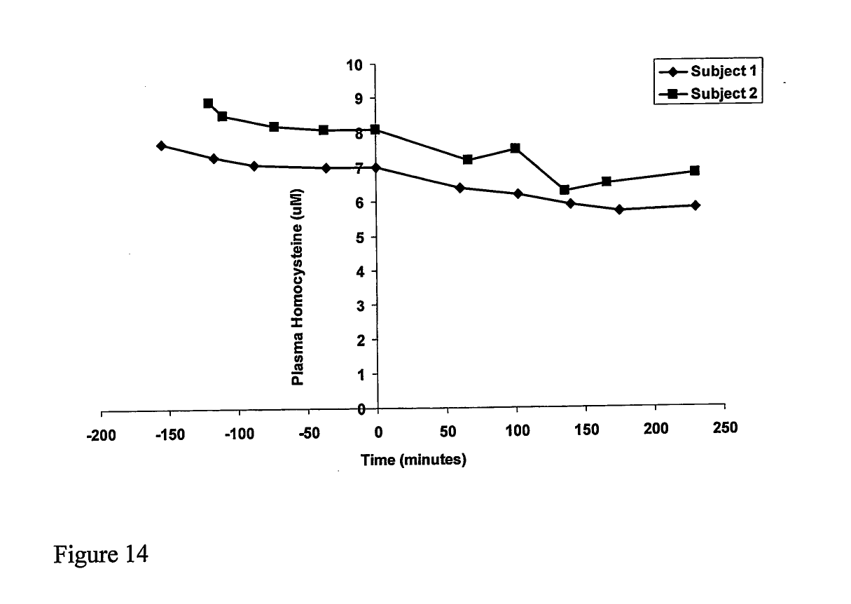

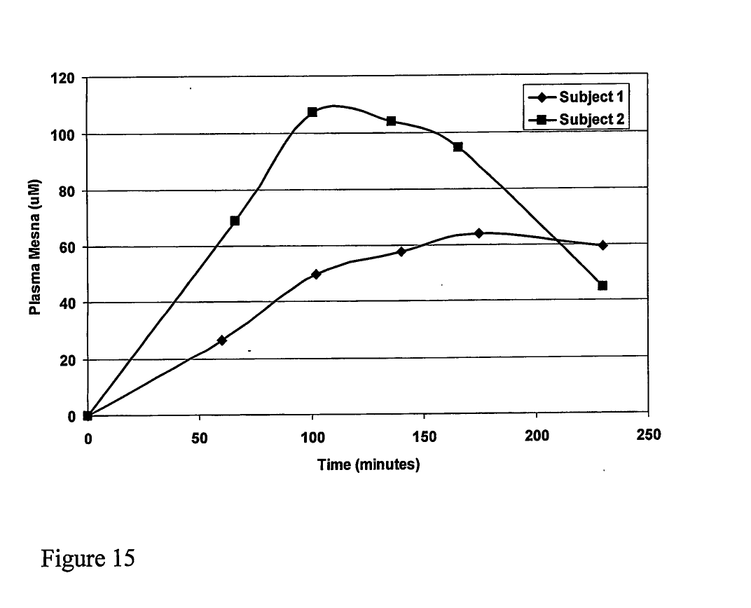

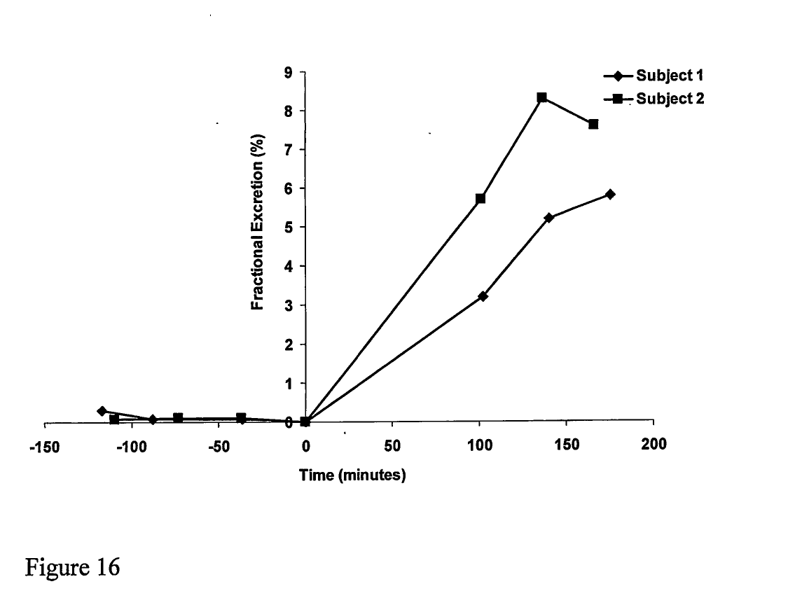

The effect of oral Mesna on Hcy clearance was also evaluated in 2 subjects with normal kidney function. The subjects fluid loaded by consuming 2 L of water the morning of the study day. An indwelling catheter was inserted into an appropriate arm vein. At the beginning of the study subjects were asked to void urine and 2×5 mL blood samples were drawn for analysis of tHcy and creatinine. Subsequent blood samples were drawn at 0.5, 1.0 and 1.5 hours for determination of baseline tHcy and creatinine. At these times subjects were also asked to void urine for measurement of urinary tHcy, creatinine and urine volume. Subjects were then given a 10 mg/kg oral dose of Mesna. One hour after the dose, subjects were asked to void their urine and 2×5 mL blood samples were drawn for determination of tHcy and creatinine. At 1.5, 2.0, and 2.5 hours after Mesna, blood samples were drawn for tHcy and creatinine analysis. Urine was also collected at these times for determination of urinary Hcy, creatinine and urinary volume. A final blood sample was drawn 4 hours after administration of Mesna. Mesna caused a slow, small (1.5 μM) decrease in plasma tHcy concentration in both subjects (see FIG. 14). Mesna was detectable in plasma however, maximum concentration (mean 88.6 μM) was less than the 5 mg/kg administered intravenously to dialysis patients (FIG. 15). The fractional excretion of Hcy ([CL_{Hcy}/CL_{Creat}]*100) was assessed in the presence and absence of Mesna (see FIG. 16). In the absence of Mesna the fractional excretion of Hcy was only 0.1%. When Mesna was administered the fractional Hcy excretion raised 60 fold to 6.0%.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR REFERENCES REFERRED TO IN THE SPECIFICATION

1. Finkelstein J D: The metabolism of homocysteine: pathways and regulation. Eur. J. Pediatr. 157 Suppl 2:S40-S44, 1998
2. Chao C L, Tsai H H, Lee C M, Hsu S M, Kao J T, Chien K L, Sung F C, Lee Y T: The graded effect of hyperhomocysteinemia on the severity and extent of coronary atherosclerosis. Atherosclerosis 147:379-386, 1999
3. Spence J D, Malinow M R, Barnett P A, Marian A J, Freeman D, Hegele R A: Plasma homocyst(e)ine concentration, but not MTHFR genotype, is associated with variation in carotid plaque area. Stroke 30:969-973, 1999
4. Vasan R S, Beiser A, D'Agostino R B, Levy D, Selhub J, Jacques P F, Rosenberg I H, Wilson P W: Plasma homocysteine and risk for congestive heart failure in adults without prior myocardial infarction. JAMA 289:1251-1257, 2003
5. Ubbink J B, Vermaak W J, van der M A, Becker P J, Delport R, Potgieter H C: Vitamin requirements for the treatment of hyperhomocysteinemia in humans. J. Nutr. 124:1927-1933, 1994
6. Hackam D G, Peterson J C, Spence J D: What level of plasma homocyst(e)ine should be treated? Effects of vitamin therapy on progression of carotid atherosclerosis in patients with homocyst(e)ine levels above and below 14 micromol/L. Am. J. Hypertens. 13:105-110, 2000
7. Anwar W, Gueant J L, Abdelmouttaleb I, Adjalla C, Gerard P, Lemoel G, Erraess N, Moutabarrek A, Namour F: Hyperhomocysteinemia is related to residual glomerular filtration and folate, but not to methylenetetrahydrofolate-reductase and methionine synthase polymorphisms, in supplemented end-stage renal disease patients undergoing hemodialysis. Clin. Chem. Lab Med. 39:747-752, 2001
8. Arnadoftir M, Hultberg B, Nilsson-Ehle P, Thysell H: The effect of reduced glomerular filtration rate on plasma total homocysteine concentration. Scand. J. Clin. Lab Invest 56:41-46, 1996
9. House A A, Donnelly J G: Effect of multivitamins on plasma homocysteine and folate levels in patients on hemodialysis. ASAIO J. 45:94-97,1999
10. Spence J D, Cordy P, Kortas C, Freeman D: Effect of usual doses of folate supplementation on elevated plasma homocyst(e)ine in hemodialysis patients: no difference between 1 and 5 mg daily.

Am. J. Nephrol. 19:405-410,1999
11. Elian K M, Hoffer L J: Hydroxocobalamin reduces hyperhomocysteinemia in end-stage renal disease. Metabolism 51:881-886, 2002
12. Bostom A G, Shemin D, Nadeau M R, Shih V, Stabler S P, Allen R H, Selhub J: Short term betaine therapy fails to lower elevated fasting total plasma homocysteine concentrations in hemodialysis patients maintained on chronic folic acid supplementation. Atherosclerosis 113:129-132,1995
13. House A A, Wells G A, Donnelly J G, Nadler S P, Hebert P C: Randomized trial of high-flux vs low-flux haemodialysis: effects on homocysteine and lipids. Nephrol.Dial.Transplant. 15:1029-1034, 2000
14. Vriese A S, Langlois M, Bernard D, Geerolf I, Stevens L, Boelaert J R, Schurgers M, Mafthys E: Effect of dialyser membrane pore size on plasma homocysteine levels in haemodialysis patients. Nephrol.Dial.Transplant. 18:2596-2600, 2003
15. Friedman A N, Bostom A G, Laliberty P, Selhub J, Shemin D: The effect of N-acetylcysteine on plasma total homocysteine levels in hemodialysis: a randomized, controlled study. Am. J. Kidney Dis. 41:442-446, 2003
16. Ventura P, Panini R, Abbati G, Marchetti G, Salvioli G: Urinary and plasma homocysteine and cysteine levels during prolonged oral N-acetylcysteine therapy. Pharmacology 68:105-114, 2003
17. Lauterburg B H, Nguyen T, Hartmann B, Junker E, Kupfer A, Cerny T: Depletion of total cysteine, glutathione, and homocysteine in plasma by ifosfamide/Mesna therapy. Cancer Chemother.Pharmacol. 35:132-136, 1994
18. Pendyala L, Creaven P J, Schwartz G, Meropol N J, Bolanowska-Higdon W, Zdanowicz J, Murphy M, Perez R: Intravenous ifosfamide/Mesna is associated with depletion of plasma thiols without depletion of leukocyte glutathione. Clin. Cancer Res. 6:1314-1321, 2000
19. Pendyala L, Schwartz G, Smith P, Zdanowicz J, Murphy M, Hausheer F: Modulation of plasma thiols and mixed disulfides by BNP7787 in patients receiving paclitaxel/cisplatin therapy. Cancer Chemother. Pharrnacol. 51:376-384, 2003
20. Jacobsen D W, Gatautis V J, Green R, Robinson K, Savon S R, Secic M, Ji J, Otto J M, Taylor L M, Jr.: Rapid HPLC determination of total homocysteine and other thiols in serum and plasma: sex differences and correlation with cobalamin and folate concentrations in healthy subjects. Clin. Chem. 40:873-881,1994
21. Bostom A G, Shemin D, Lapane K L, Miller J W, Sutherland P, Nadeau M, Seyoum E, Hartman W, Prior R, Wilson P W.: Hyperhomocysteinemia and traditional cardiovascular disease risk factors in end-stage renal disease patients on dialysis: a case-control study. Atherosclerosis 114:93-103, 1995
22. Bostom A G: Homocysteine: “expensive creatinine” or important modifiable risk factor for arteriosclerotic outcomes in renal transplant recipients? J. Am. Soc. Nephrol. 11:149-151, 2000
23. Ducloux D, Aboubakr A, Mofte G, Toubin G, Fournier V, Chalopin J M, Drueke T, Massy Z A: Hyperhomocysteinaemia therapy in haemodialysis patients: folinic versus folic acid in combination with vitamin B6 and B12. Nephrol. Dial. Transplant. 17:865-870, 2002
24. Sigit J I, Hages M, Brensing K A, Frotscher U, Pietrzik K, von Bergmann K, Lutjohann D: Total plasma homocysteine and related amino acids in end-stage renal disease (ESRD) patients measured by gas chromatography-mass spectrometry—comparison with the Abbott IMx homocysteine assay and the HPLC method. Clin. Chem. Lab Med. 39:681-690, 2001
25. Soria C, Chadefaux B, Coude M, Gaillard O, Kamoun P: Concentrations of total homocysteine in plasma in chronic renal failure. Clin. Chem. 36:2137-2138,1990
26. Souid A K, Fahey R C, Aktas M K, Sayin O A, Karjoo S, Newton G L, Sadowitz P D, Dubowy R L, Bernstein M L: Blood thiols following amifostine and Mesna infusions, a pediatric oncology group study. Drug Metab Dispos. 29:1460-1466, 2001
27. Yamamoto N, Tanaka T, Noguchi T: Effect of cysteine on expression of cystathionine beta-synthase in the rat liver. J. Nutr. Sci. Vitaminol. (Tokyo) 41:197-205,1995
28. Goren M P, Hsu L C, Li J T: Reduction of diMesna to Mesna by the isolated perfused rat liver. Cancer Res. 58:4358-4362, 1998
29. Friedman A N, Bostom A G, Levey A S, Rosenberg I H, Selhub J, Pierratos A: Plasma total homocysteine levels among patients undergoing nocturnal versus standard hemodialysis. J. Am. Soc. Nephrol. 13:265-268, 2002.
30. Nygard O, Nordrehaug J E, Refsum H, Ueland P M, Farstad M, Vollset S E: Plasma homocysteine levels and mortality in patients with coronary artery disease. N. Engl. J. Med. 337:230-236, 1997.
31. Wald D S, Law M, Morris J K: Homocysteine and cardiovascular disease: evidence on causality from a meta-analysis. BMJ 325:1202, 2002.

1. A method of lowering elevated plasma total homocysteine (tHcy) levels in a subject with end stage renal disease comprising administering an effective amount of Mesna, or a derivative thereof, to a subject having end stage renal disease (ESRD).

2. The method according to claim 1, wherein the derivative of Mesna is diMesna.

3. The method according to claim 1, wherein by lowering the tHcy levels in the plasma of a patient with ESRD, the risk of cardiovascular-related diseases is also reduced.

4. The method according to claim 3, wherein the cardiovascular-related disease is selected from myocardial infarction, stroke, thrombosis and atherosclerosis,

5. The method according to claim 4, wherein thrombosis is a thrombotic event selected from venous thrombosis, dialysis access thrombosis and thrombotic stroke.

6. The method according to any one of claim 1, further comprising performing dialysis on the subject.

7. The method according to claim 5, wherein the dialysis is performed during or subsequent to administration of Mesna or derivative thereof.

8. The method according to any one of claim 1, wherein the subject is human.

9. The method according to any one of claim 1 wherein Mesna, or a derivative thereof, is administered at a dosage of about 0.5-180 mg/kg per week.

10. The method according to claim 8, wherein Mesna, or a derivative thereof, is administered at a dosage of about 1.0-25 mg/kg per week.

11. The method according to claim 9, wherein Mesna, or a derivative thereof, is administered at a dosage of about 7.5-15 mg/kg per week.

12. The method according to claim 8, wherein Mesna, or a derivative thereof, is administered at a dose of between about 2.5 to 5 mg/kg thrice weekly.

13. The method according to claim 1 wherein Mesna, or a derivative thereof, is administered intravenously or orally.

14. The method according to claim 1, wherein Mesna, or a derivative thereof, is administered in combination with other agents that lower plasma thiol levels or in combination with other types of treatment for diseases associated with elevated plasma thiol levels.

15. The method according to claim 13, wherein Mesna, or a derivative thereof, is administered in combination with B vitamins and/or folic acid

16-18. (canceled)
